(19) United States
(12) Patent Application Publication
(10) Pub. No.: US 2002/0134378 A1
(43) Pub. Date: Sep. 26, 2002

(54) Sound dampening housing for respiratory assist devices

(76) Inventors: Linda A. Finnegan, Phoenix, AZ (US); James E. Hawthorne, Calgary (CA)

Correspondence Address: ROSENBAUM & ASSOCIATES, P.C., Suite 3653, 875 North Michigan Avenue, Chicago, IL, 60611, US

(21) Appl. No.: 09/817,476

(22) Filed: Mar. 26, 2001

(51) Int. Cl. A62B 7/00; A62B 9/00; A61M 15/00; A61M 16/00; A62B 18/00

(52) U.S. Cl. 128/200.24

(57) ABSTRACT

A sound dampening housing for conventional respiratory assist devices fabricated of pliant synthetic materials that provide good wear life and cleanability, and provide excellent sound dampening properties. The sound dampening housing substantially encloses a conventional respiratory assist device, but has openings or openable sections which positionally correspond to the functional elements of the respiratory assist device, e.g., air hose connections, blower or pump intakes, control interfaces, power connectors, etc.

### BACKGROUND OF THE INVENTION

The present invention relates generally to housings for respiratory devices. More particularly, the present invention relates to sound dampening housings for respiratory devices, such as continuous positive airway pressure (CPAP) apparatus', inspiratory positive airway pressure (IPAP) apparatus', expiratory positive airway pressure (EPAP) apparatus, and other respiratory apparatus' which employ a pump to expel an air volume and direct the air volume into a person's trachea to assist the person in breathing.

A plethora of various respiratory assist devices are available in the marketplace. For example, different respiratory assist device models are available from DeVilbiss Health Care, Inc./Sunrise Medical, Inc. (Fort Pierce, Fla.), ResCare, Inc. (San Diego, Calif.), ResMed, Limited (Abingdon, Oxon, U.K.), and Respironics, Inc. (Forest Hills, Pa.) manufacture and market a wide variety of CPAP, IPAP, EPAP and bi-CPAP devices for use by people suffering from obstructive sleep disorders, particularly sleep apnea. Each of these conventional devices shares certain common essential functional elements. Specifically, these devices include a blower or pump that generates positively pressurized air, a mask or nasal cannula and a hose interconnecting the blower or pump and the mask of cannula to deliver positively pressurized air to the person's respiratory system. Conventional systems also include a pressure controller which measures the pressure of the delivered air and compares the measured pressure with a stored or set pressure and adjusts the pressure, such as by controlling the speed of the blower or pump, to correct for any deviations between the measured pressure and the preset pressure. Examples of conventional respiratory assist devices specifically for treating obstructive sleep Pat. No. Re. 35,295, U.S. Pat. No. 5,865,173, U.S. Pat. No. 4,655,213 and U.S. Pat. No. 4,773,411.

While great advances have been made in fabricating respiratory assist devices which have sound dampening to minimize audible noise generated by the device, particularly by the blower or pump, it has been found that many conventional devices suffer from unacceptably high noise levels in frequency ranges which human beings are particularly susceptible to sensing. Accordingly, in order to further reduce the audible noise emitted by respiratory assist devices it has been found desirable to provide an external sound dampening housing which surrounds a respiratory assist device and permits the functional elements of the respiratory assist device, e.g., the air hose, the blower or pump intake, the control interface, to be exposed or accessible to the user. By providing a sound dampening housing, the present invention significantly reduces audible noise emissions from conventional respiratory assist devices.

### SUMMARY OF THE INVENTION

It is a primary objective of the present invention to provide a sound dampening housing for conventional respiratory assist devices. The inventive sound dampening housing is fabricated of pliant synthetic materials that provide good wear life and cleanability, and provide excellent sound dampening properties. The inventive sound dampening housing substantially encloses a conventional respiratory assist device, but has openings or openable sections which positionally correspond to the functional elements of the respiratory assist device, e.g., air hose connections, blower or pump intakes, control interfaces, power connectors, etc.

The inventive sound dampening housing of the present invention consists generally of an enclosure formed from sections of vinyl film laminated onto a closed-cell foam material. A plurality of sections of foam-vinyl laminate material are joined, such as with seam stitching, or heat sealing, to one another to form an enclosure having a geometry which conforms to the outer dimensions of any given respiratory assist device. Because virtually all respiratory assist devices have generally cubic or rectilinear geometries, the inventive sound dampening enclosure will typically be a six-sided enclosure housing, with each side of the enclosure housing conforming to and corresponding with one geometric aspect of the pre-existing respiratory assist device. Thus, for example, since most conventional respiratory assist devices have top and bottom surfaces, two lateral side surfaces and front and rear surfaces, the inventive sound dampening housing will have corresponding top and bottom surfaces, two lateral side surfaces and front and rear surfaces, thereof.

Because pre-existing respiratory assist devices have their functional elements positioned differently, the inventive sound dampening enclosure will have corresponding openings passing through the surfaces thereof in order to accommodate access to or passage of functional elements through surfaces forming the sound dampening enclosure. For example, where one CPAP device may have an air intake opening on a front surface of the device, and hence the inventive sound dampening enclosure would have a correspondingly dimensioned opening on its front surface, another CPAP device may have its air intake opening on a lateral side surface thereof. In this latter case, the inventive sound dampening enclosure would be configured to have a correspondingly dimensioned and positioned opening on the corresponding lateral side surface of the sound dampening enclosure. One of ordinary skill in the art will understand and recognize that it is within the skill of the artisan to configure the inventive sound dampening enclosure with a geometry which conforms to a particular pre-existing respiratory assist device and have openings passing through surfaces thereof which both positionally and dimensionally correspond to the functional elements of a given pre-existing respiratory assist device.

### BRIEF DESCRIPTION OF THE DRAWINGS

FIG. 1 is a perspective view of the inventive sound dampening enclosure for a respiratory assist device.

FIG. 2 is a cross-sectional view taken along line 2-2 of FIG. 1.

FIG. 3 is a cross-sectional view taken along line 3-3 of FIG. 1.

FIG. 4 is a cross-sectional view taken of section 4 is FIG. 2.

FIG. 5 is a perspective view of the inventive sound dampening enclosure with a respiratory assist device therein in partial phantom.

FIG. 6 is a graph illustrating the sound dampening effect of the inventive sound dampening enclosure when used with a CPAP respiratory assist device.

### DETAILED DESCRIPTION OF THE PREFERRED EMBODIMENTS

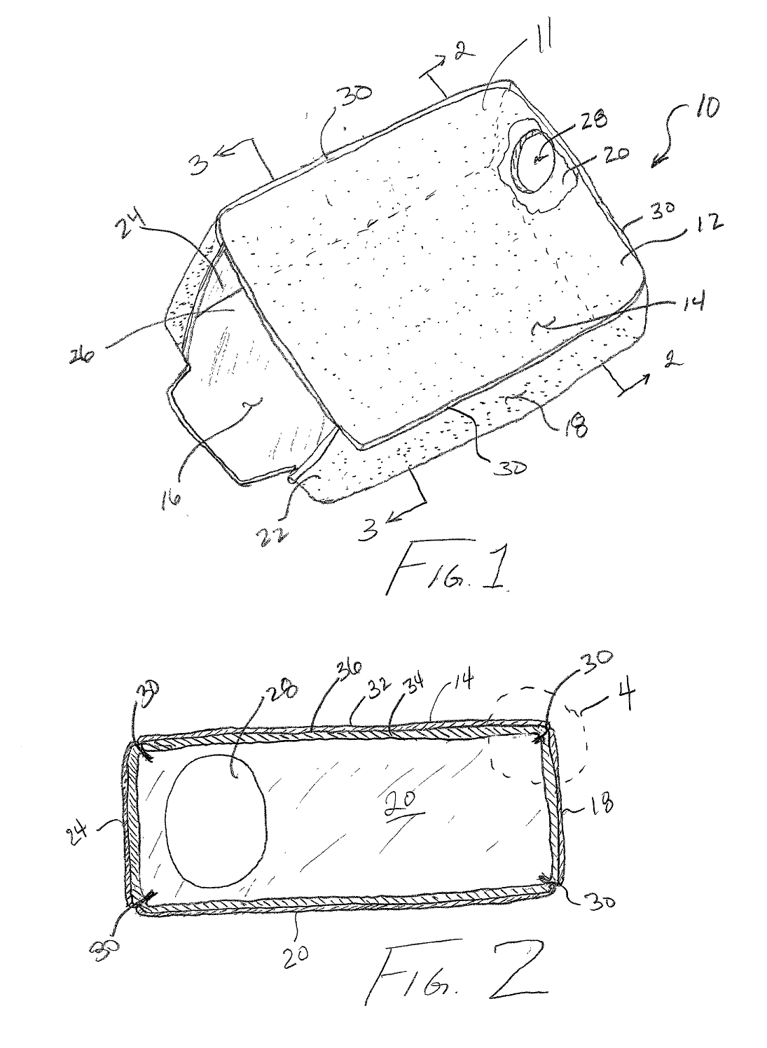

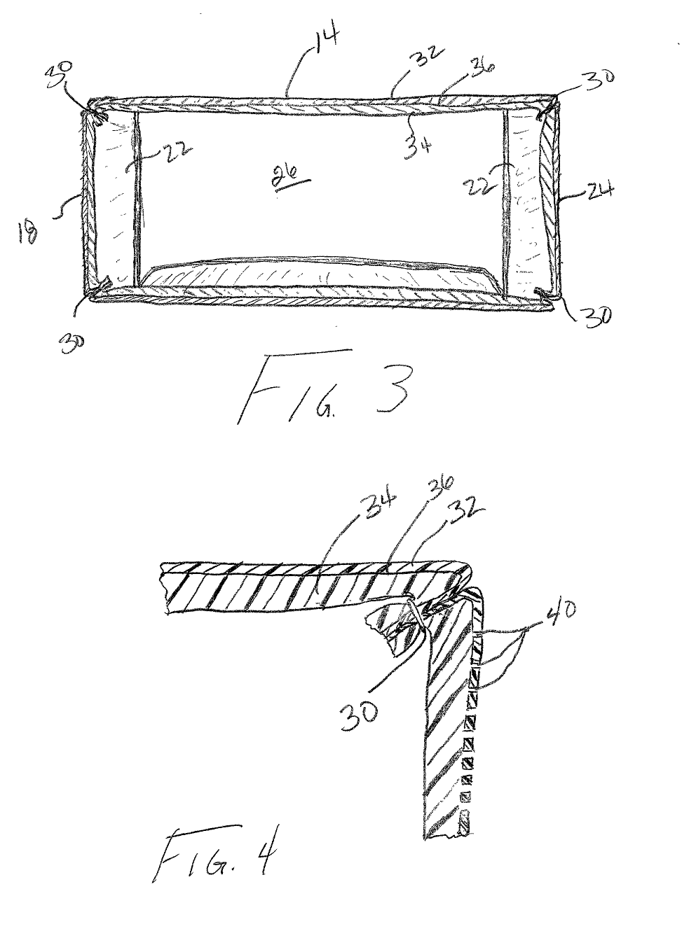

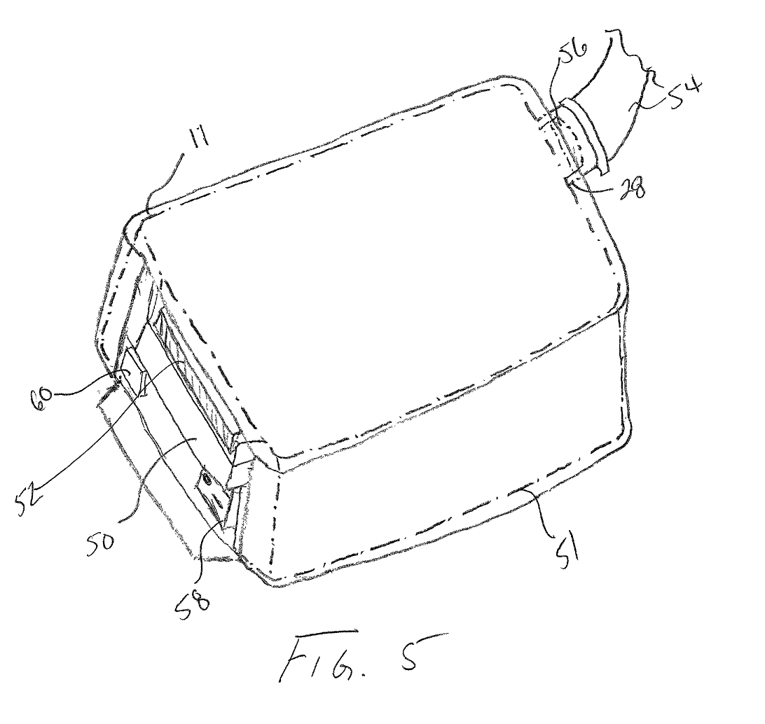

The inventive sound dampening enclosure 10 is illustrated in FIGS. 1-5 and the degree of sound dampening effect is illustrated in the graph at FIG. 6. In the accompanying figures, like reference numerals in each figure denote like elements. The inventive sound dampening enclosure 10 for a respiratory assist device consists generally of a three-dimensional housing 11 formed from plurality of panel members 12 joined together by at least one of a plurality of seams 30. In order to provide acceptable sound dampening to a respiratory assist device that is contained within the housing 11, each of the plurality of panel members 12 is fabricated of a material that is pliant, easy to clean, durable and is capable of attenuating sound transmission from the respiratory assist device. In accordance with a preferred embodiment of the present invention, the plurality of panel members 12 are fabricated of a plastic film 32 laminated to a foam backing 34 by an adhesive interlayer 36. The foam backing is preferably either a closed-cell or open-cell foam, and is most preferably a closed-cell foam. The foam backing preferably has a z-axis thickness between ⅛^{th} inch (0.3175 cm) to 1 inch (2.54 cm), and most preferably has az-axis thickness of between 0.25 inch (0.635 cm) to 0.5 inch (1.27 cm). Preferable foam materials are polyurethane foams. The plastic film may have a plurality of perforations 40 passing there through or may be imperforated. Additionally, the plastic film 32 may include decorative patters embossed therein. Preferred materials for fabricating the plastic film 32 include vinyl and polyester materials. Alternatively, fabric materials may be substituted for the plastic film 32. Vinyl film-foam laminate materials which have been found especially useful in the present invention are those which are currently used in the automotive industry to form headliners for passenger compartment roofs. Various configurations of plastic film-foam laminate materials are known in the art as represented by U.S. Pat. Nos. 5,582,906, 4,150,850, 5,965,251, 5,952,089, and similar patents. Commercially available examples of vinyl-closed cell foam materials which are suitable for use with the present invention are sold by GAHH, Inc., (8116 Lankershim Blvd, North Hollywood, Calif. 91605), Gilbreath Upholstering and Auto Trimming Supply, Inc. (Des Moines, Iowa), American Trim & Upholstery (1355 N. Marion, Tulsa, Okla. 74158) and a vinyl-foam laminate material sold under the trademark VELOCITY by J. Ennis Fabrics, Ltd. (12122-68 Street, Edmonton, Alberta, Canada).

The vast majority of commercially available respiratory assist devices have generally polygonal geometries. However, certain respiratory assist devices have generally cylindrical geometries. While the sound dampening enclosure 10 of the present invention will be described with reference to a sound dampening enclosure 10 having polygonal geometry, it will be understood by those of ordinary skill in the art that the particular geometry of the inventive sound dampening enclosure 10 will vary depending upon the particular model of respiratory assist device intended to be retained within the sound dampening enclosure 10.

In accordance with a preferred embodiment of the present invention, there is provided a sound dampening enclosure 10 in which the plurality of panel members 12 are joined together by at least one of a plurality of seams into a polygonal geometry which defines a top surface 14, bottom surface 16, first side surface 18, second side surface 24, rear surface 20 and frontal surface 22 of the three-dimensional housing 11. The at least one of a plurality of seams 30 may be formed by a plurality of sewing stitches, heat welding, adhesive interconnection, or other suitable method of joining the plurality of panel members 12. It will be understood by those of ordinary skill in the art that different respiratory assist devices will have their functional elements arranged and positioned differently from other respiratory devices. Similarly, the dimensioning and configuration of the functional elements of a given respiratory assist device will be different from other respiratory assist devices. As noted above, by their nature, most respiratory assist devices have common functional elements, e.g., a blower or pump that generates positively pressurized air, a mask or nasal cannula, a hose interconnecting the blower or pump and the mask of cannula to deliver positively pressurized air to the person's respiratory system, an electrical control interface and an electrical power connector. In order to accommodate these functional elements of conventional respiratory assist devices, the inventive sound dampening enclosure 10 includes at least one of a plurality of openings 26, 28 which both positionally and dimensionally correspond to the position and dimension of the functional elements of the respiratory assist device.

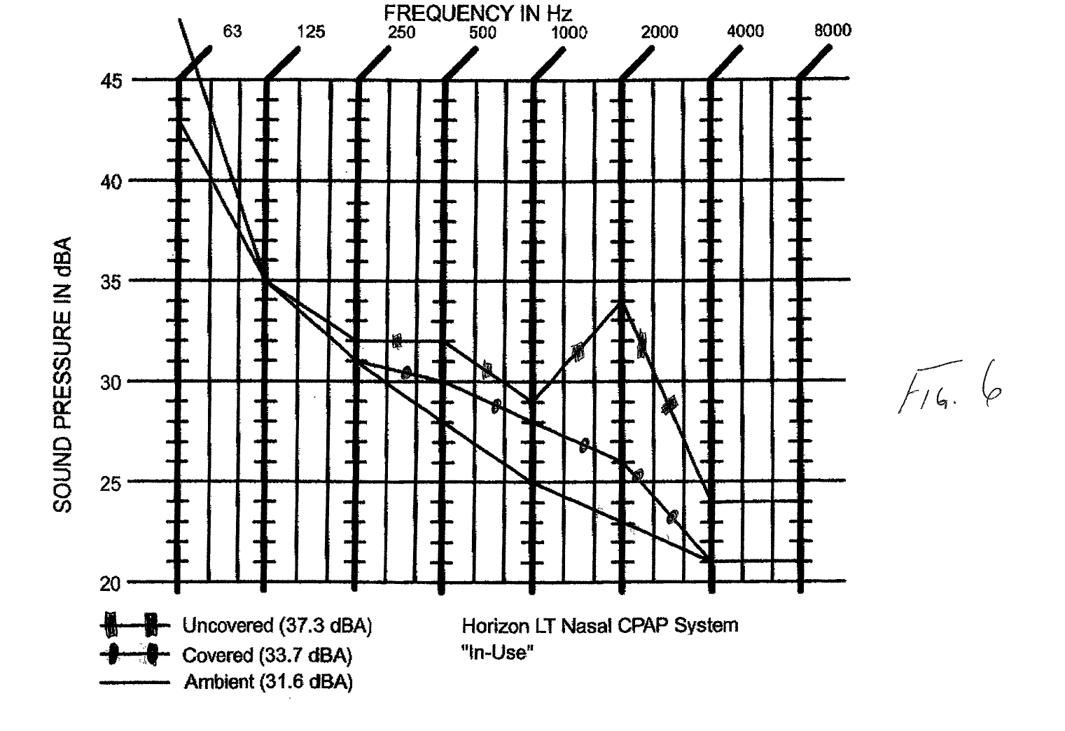

By way of a non-limiting example, the present invention is depicted in the accompanying figures with geometry and configuration suitable for use with a CPAP respiratory assist device sold under the trademark HORIZON LT NASAL CPAP SYSTEM (Sunrise Medical, Respiratory Products Division, Somerset, Pennsylvania). As illustrated in FIG. 5, the HORIZON LT NASAL CPAP SYSTEM 50, which is depicted enclosed within the inventive sound dampening enclosure housing 11, consists generally of a housing 51 which contains the functional elements of the device, but has a power switch 60, an AC power connector 58, and an air-inlet port 52 all positioned on a first end surface of the CPAP system 50. An air outlet port 56, and air tubing 54 connected to the air outlet port 56 are positioned on a second end surface of the CPAP system 50 which opposes the first end surface of the CPAP system 50. Given the particular geometry of the CPAP system 50, as illustrated in FIGS. 1-5, the inventive sound dampening enclosure housing 11 has an opening 28 which passes through the panel member 12 which forms the rear surface 20 of the housing 11. Opening 28 is positioned and dimensioned, in this example, to both positionally and dimensionally correspond to the position and dimension of the air outlet port 56 1-5 of the CPAP system 50 and permit passage of air outlet port 56 through opening 28.

Similarly, because in the CPAP system 50, all other functional elements of the system 50, i.e., the power switch 60, AC power connector 58 and air-inlet port 52, are positioned on the first end surface of the CPAP system 50, the inventive sound dampening enclosure housing 11 has a second opening 26 in a front surface 22 of the housing 11 which is both positioned and dimensioned to permit the corresponding functional elements of the system 50 to be accessed there through. The number, position and dimension of the at least one of a plurality of openings 26, 28 in the sound dampening enclosure housing 11 will, of course, correspond to the number, position and dimension of the functional elements requiring access through the plurality of panel members 12 forming the sound dampening enclosure housing 11.

Most respiratory assist devices, and in particular, those respiratory assist devices, which are used by people having obstructive sleep disorders such as sleep apnea, are intended for use while sleeping. While many conventional respiratory assist devices are designed to have very low audible noise emissions, human beings are particularly sensitive to sound having frequencies within the range of 2,000 to 4,000 Hz. Thus, it is particularly important to attenuate audible noise occurring in the 2,000 to 4,000 Hz frequency range.

In order to test the effectiveness of the present invention in attenuating audible noise, a sound dampening enclosure was fabricated for the HORIZON LT NASAL CPAP SYSTEM, as illustrated in FIGS. 1-5. The sound dampening enclosure was fabricated of a plurality of panel members 12 each fabricated of a perforated vinyl film outer layer laminated onto a closed cell polyurethane foam layer having a z-axis thickness of 0.5 inches (1.27 cm). The plurality of panel members 12 were joined together to form a hexahedral enclosure having a major top surface, a major bottom surface, a rear surface, first and second side surfaces and a front surface. The rear surface, first and second side surfaces and the front surface panel members 12 were joined to the top surface and bottom surface by sew stitched seams which resided on an inner aspect of the main enclosure chamber formed by the hexahedral enclosure.

In order to determine the effective sound attenuation capability of the inventive sound dampening enclosure, sound measurements were performed with a Type 1 sound lever meter and a spectrum analyzer. A-weighted sound levers were measured which measured sound pressure level, filtered or weighted at various frequencies to approximate the response of the human ear. Measurements were taken of the ambient environment, the HORIZON LT NASAL CPAP SYSTEM operating without the inventive sound dampening enclosure, and of the HORIZON LT NASAL CPAP SYSTEM operating within the inventive sound dampening enclosure. Measurements were made across the frequency range of 63 to 8000 Hz. The resulting measurements of the operating HORIZON LT NASAL CPAP SYSTEM, both with and without the inventive covering, were less than 9 dB above the existing ambient noise level. The measurements were, therefore biased or overstated by mathematically backing out the background noise level at each individual frequency to approximate the results of laboratory measurements. The corrected uncovered equipment sound level was a maximum of 36.3 dBA while the corrected covered equipment sound level was a maximum of 29.8 dBA. The resulting reduction of 6.5 dBA between the covered CPAP system and the uncovered CPAP system represents a clearly noticeable change in apparent loudness of the covered CPAP system relative to the uncovered CPAP system. The sound attenuation effect of the inventive sound dampening enclosure is graphically illustrated in FIG. 6 which demonstrates a significant reduction in audible nose transmission in the 2,000-4,000 Hz frequency range of the covered CPAP system versus the uncovered CPAP.

While the present invention has been described with reference to its preferred embodiments, those of ordinary skill in the art will understand and appreciate that variations in structural geometry, material selection, and position and dimension are regarded within the skill of the artisan and that alternative constructions and materials are contemplated by the present invention which is limited only by the claims appended hereto.

What is claimed is:

1. A sound dampening enclosure for a respiratory assist device, comprising a plurality of panel members, each fabricated from a pliant material having sound dampening properties, each of the plurality of panel members being joined together to form an enclosure having a three-dimensional geometry, and at least one opening in the sound dampening enclosure defining a passageway for a functional element of the respiratory assist device.

2. The sound dampening enclosure according to claim 1, wherein the plurality of panel members are each fabricated of a plastic film laminated to a foam material.

3. The sound dampening enclosure according to claim 2, wherein the plastic film is selected from the group of vinyl and polyethylene.

4. The sound dampening enclosure according to claim 2, wherein the plastic film further comprises an perforated vinyl film.

5. The sound dampening enclosure according to claim 2, wherein the foam material is selected from the group of open-cell and closed-cell foam materials.

6. The sound dampening enclosure according to claim 2, wherein the plastic film component of the plastic film-foam laminate material forms all exterior surfaces of the sound dampening enclosure.

7. The sound dampening enclosure according to claim 5, wherein the plastic film is a vinyl film and the foam material is a closed-cell foam material, wherein the vinyl film forms all exterior surfaces of the sound dampening enclosure and the closed-cell foam material forms substantially all inner surfaces of the sound dampening enclosure.

8. The sound dampening enclosure according to claim 1, wherein the three-dimensional geometry further comprises a polyhedron.

9. The sound dampening enclosure according to claim 8, wherein the polyhedron further comprises a hexahedron.

10. The sound dampening enclosure according to claim 8, wherein the polyhedron defines a plurality of planar surfaces of the sound dampening enclosure formed by planar members defining at least a top surface, a bottom surface, a first side surface, a second side surface, a frontal surface and a rear surface.

11. The sound dampening enclosure according to claim 10, wherein the at least one opening passes through at least one of the plurality of planar surfaces of the polyhedron.

12. The sound dampening enclosure according to claim 1, wherein the at least one opening is formed by non-joined portions of the plurality of planar members.

13. The sound dampening enclosure according to claim 10, wherein the rear surface has at least one opening passing there through that accommodates an air outlet of a respiratory assist device.

14. The sound dampening enclosure according to claim 13, further comprising a frontal surface opening defined by non-joined ends of the planar members first side surface and the second side surface which are continuous with the frontal surface and by frontal edges of each of the top surface and the bottom surface.

15. The sound dampening enclosure according to claim 14, wherein the frontal surface opening is positioned and dimensioned to accommodate access to at least one of an air intake, an electronic control panel and an electrical power connector.

16. A sound dampening enclosure for a respiratory assist device, comprising a plurality panel members fabricated from a foam and vinyl film laminate material, each of the plurality of panel members being joined together to form a polyhedral enclosure having a plurality of outer surfaces and defining an interior chamber for receiving the respiratory assist therein, at least on of the plurality of outer surfaces having at least one opening permitting access to at least one functional element of the respiratory assist device.

17. The sound dampening enclosure according to claim 16, wherein the foam component of the foam and vinyl film laminate material is selected from the group consisting of open cell foam and closed cell foam.

18. The sound dampening enclosure according to claim 16, wherein the vinyl film further comprises a perforated vinyl film.

19. The sound dampening enclosure according to claim 16 wherein each of the plurality of panel members are joined by seam stitching at abutting edges thereof.

20. The sound dampening enclosure according to claim 16, wherein the polyhedral enclosure comprising at least a top surface, a bottom surface, a first side surface, a second side surface, a frontal surface and a rear surface.
